(19) United States
(12) Patent Application Publication
(10) Pub. No.: US 2007/0016271 A1
(43) Pub. Date: Jan. 18, 2007

(54) Spot heating system for human and animal use

(76) Inventor: Mike H. Hammond, Seattle, WA (US)

Correspondence Address: MICHAEL H. HAMMOND, 4841 CALIFORNIA AVE. S.W., SEATTLE, WA, 98116, US

(21) Appl. No.: 11/182,966

(22) Filed: Jul. 15, 2005

(51) Int. Cl. A61F 7/00; A61F 7/12

(52) U.S. Cl. 607/96

(57) ABSTRACT

A heating pad adapted for heating specific portion of the body of a human or animal is disclosed. The element is PTC temperature self-regulating and operates on low voltage, therefore may be used by invalids, sleeping persons, and others without danger of scalding or setting fire. Other uses for the basic heating pad system are described.

### CROSS-REFERENCE TO RELATED APPLICATION

This application claims the benefit of Provisional Application No. 60/499,813, filed Sep. 3, 2003, and U.S. patent application Ser. No. 10/930,106, the benefit of which are hereby claimed under 35 U.S.C. section 119.

application Ser. No. 10/930,106, is hereby incorporated by reference.

### FIELD OF THE INVENTION

The present invention relates to portable, wearable heating pads adapted for spot heating of the anatomy of persons and animals.

### U.S. GOVERNMENT INTEREST IN THE INVENTION

None.

### BACKGROUND OF THE INVENTION

1. Field of the Invention

Self regulating spot heaters for therapeutic and comfort uses.

2. Description of Related Art

Spot heaters for localized heating of human and animal skin or hide are either chemically operated heating pads or electrically operated. Chemical pads are one-time use, present disposal problems, and can injure the user or others if the chemical packet is ruptured. Furthermore, there is no temperature control system other than user vigilance for overheating and removing the pad.

Electrically operated pads are equipped with wire type heating elements and usually operate on utility electrical service. There are several serious problems with such construction. The least dangerous is that the wires break easily. In use, wire heaters will continue to absorb full energy even though some or all of the pad is covered or folded which prevents the escape of the heat generated. Generally one or more temperature sensors are installed in series with the wire elements to disrupt the current if, and only if, one of the sensors detects over heating. The sensor(s) may be in a portion of the pad that is not covered and is not over heating. This is obvious in the case of electric blankets. A pillow, book, newspaper, etc in the center of a blanket will cause local overheating and may not be detected. Smaller pads tend to have significant wattage and only rudimentary control by manually switching a plurality of heater segments on and off. Because of these adverse characteristics, the instructions for electrical heating pads always stress that they are unsuitable for use on persons that may fall asleep under them, or who have sensory problems such as with diabetes and cannot feel excessive temperature.

Also, some people are concerned about the electromagnetic field generated by current flowing in the serpentine path of heating wires.

Controllers for precise temperature regulation of wire heating pads are described in several patents. This control relies on sensitive, complicated circuitry to measure the small changes in resistance of the heating wires caused by the temperature of the wires. The resistance measurement is then used to operate an energy modulator to control the average temperature. A hot spot similar to that which has been previously described may or may not have a local change of resistance adequate to be sensed when averaged with the lower temperature segments of the heating elements. In general, metals have a very small temperature coefficient of resistivity. The very durable alloys designed for resistance heating wires have the lowest coefficient of all common metals and alloys; from zero to 1/20 of the coefficient for copper.

There is a class of electrical conductors possessing a very large positive temperature coefficient (PTC) of resistance, which means that the element resistance increases dramatically with increasing temperature. When a constant voltage is presented to a length of such a conductor the resistance rises with the temperature, thus reducing the flow of current, which correlates directly with reduced energy converted to heat. Thus, the product is self regulating in temperature. One such product in the form of flexible polymer plastic sheeting is described in U.S. Pat. No. 5,696,869 by Stephan Irgens, and is available from Electro-Plastics, Inc. of Saint Louis, Mo.

Irgens' conductive polymer changes resistance by a factor of approximately 1.8 over the temperature range of interest (90 to 120F) for the present invention, which is sufficient to support adequate self-regulation of temperature within the target range. The larger the factor, the better the self regulation property is exhibited. The resistance change with temperature is approximately a constant amount/degree [or (1+100*percentage)/degree]. The idealized formula for resistance change is a semi-logarithmic function in the form R=RO(kˆt) The conductive sheeting is in effect a myriad of parallel heating conductors, which effectively eliminates disabling due to conductor breakage. Therefore the conductive polymer sheet can be perforated to pass perspiration or for construction processes. The temperature set-point is determined by a combination of the composition of the sheeting, the distance between the power distribution busses, the voltage applied, and the rate of heat transfer to the subject. The product can be produced as sheets, ribbons, or bands, and is usually operated on low voltages for example, 2 to 24 volts. For these, and other, reasons, conductive polymer sheeting as described is ideal for portable, wearable heating pads.

Stephan Irgens' patent, U.S. Pat. No. 5,696,869, is incorporated herein by reference.

U.S. Pat. No. 5,371,340 by Philip Stanfield, describes a heating pad using wire elements and a thermostat switch for control. Stanfield's thermostat has a hysteresis of 15F degrees, which is unacceptable for the uses of the present invention. Plus, Stanfields invention has the potential for spot overheating as cited above for wire elements. In addition, Stanfield discloses insulation layer of bubble wrap and reflective foil to over come the back-side losses common in prior art heating pads. Furthermore, Stanfield's pad for use as a mat by hogs presents a severe fire and shock hazard to the hogs. Hooves and teeth can break into the mat and power wiring, and a break in the heater wires can cause arcing which could burn through the protective layers. The Stanfield disclosed invention has no control or prevention of local, over heating or cooling as does the present invention. Urine, milk, or other liquids can cause local cold spots, and the insulating properties of the animal body will promote increased localized heating under the animal. The heating element of the present invention has operating characteristics to regulate heat production on a localized basis.

Stanfield does not disclose the use of a PTC conductive sheet heating element. Mere substitution of a conductive polymer sheet for the heating elements and thermostat would substantially alter the mode and way Stanfield's invention works,, therefore Stanfield should not be cited as prior art.

U.S. Pat. No. 5,897,162 by Linda Humes, et al, discloses a seat for an infant having a single heating wire disposed in a serpentine pattern powered by a battery pack, and a thermal switch for temperature control. Humes' back padding (30) is primarily for seating comfort of the infant, any thermal insulation is incidental and not disclosed. In contrast to the present invention and Stanfield, there is no disclosure of any insulation provided specifically to control heat flow in any direction. The present invention has such specific flow control insulation to prevent energy waste and therefore should be much more efficient than Hume's disclosed invention. The Hume invention is also susceptible to local and general overheating because of heat retention by blankets, the infant's clothing, books, paper goods, some toys, and other things likely to be in the seat with the infant or small child.

Hume does not disclose the use of a PTC conductive sheet heating element or substantial energy loss management. Mere substitution of a conductive polymer sheet for the heating elements and thermostat would substantially alter the mode and way Stanfield's invention works, therefore Hume should not be cited as prior art.

Neither should a combination of Stanfield and Hume be cited as neither can function in the manner disclosed with PTC polymer heating elements substituting for the wire elements. Adding a battery pack to Stanfield would require a complete redesign of his heater element to utilize battery voltages.

U.S. Pat. No. 5,643,480 by Magnus Gustavsson discloses a battery operated heating system utilizing PTC material for temperature regulation. One of the elements in Gustavasson's system making use of the heat generated is the battery itself, which is susceptible to failure or reduced capacity when chilled. Gustavasson discloses no insulation on the distal side of his heater. Gustavasson's battery is also a series of film layers and is constructed integrally with the heating element as a flexible assembly.

In one embodiment, Gustavasson places a reflective layer as the exterior layer of the battery. The reflective layer is not part of a composit insulating layer as is the reflecting layer in the present invention. No reflectors are disclosed as part of any insulating layer (no insulating layer is disclosed) or general heat flow control means.

All of the layers of both the battery and heater are very thin, so that when the many layers are laminated together, the assembly is still flexible enough to be used like cloth. In contrast, the conductive polymer heater element of the present invention is quite thick, approximately 0.05 inches. The thinness and layered structure of the Gustavasson assembly dictates that the terminal electrodes on the heating layer be on the front and back surfaces. In contrast, the present invention uses edge electrodes which cause the current to flow from edge to edge. The two modes are mutually exclusive.

Because substituting a PTC sheet heater for the wire heaters of Stanfield and Hume would alter the way and mode of the Stanfield and Hume inventions, PTC sheet heaters should not be combined with Stanfield or Hume to form 35USC 103 objections. PTC materials have, and are used in the present invention, characteristics different from metallic heating elements not anticipated, disclosed, or utilized by Stanfield or Hume.

Neither of the prior art references suggest the desirability of controlling localized over temperature due to miscellaneous thermal insulation from blankets or other items expected to be present during use. Nor is the problem of local over heating recognized by the references. The PTC heating element of the present invention does control localized overheating. Wire heating elements do not.

### OBJECTS OF THE PRESENT INVENTION

It is an object of the present invention to provide a spot heater for heating animal tissue for relief of pain, wound healing, comfort, treating hypothermal trauma, and the like.

It is another object of the invention to provide for safe operation of the invention by utilizing an inherent characteristic of the construction materials that controls and limits the temperature to approximately 101 F. degrees.

It is another object of the invention to provide for safe operation of the invention by utilizing an inherent characteristic of the construction materials that permits the invention to be operated on voltages under 24 volts.

It is another object of the invention to provide for safe operation of the invention by utilizing an inherent characteristic of the construction materials that permits the tissue covered by the invention to “breathe”.

It is another object of the invention to provide means for wearing the invention on the person.

It is another object of the invention to operate the heater on battery power.

### BRIEF SUMMARY OF THE INVENTION

A low voltage heating element having the a strong positive temperature coefficient of resistance (PTC) is incorporated in a pad that can be inserted into a pocket on a vest, belt, cushion, or similar structure so that the pad may be placed in close proximity of the user's skin. In particular, along the spine and over the muscles of the lower back, or around portions of the limbs, ears, chest, abdomen, buttocks, and over the body locations where major veins or arteries are near the surface, etc.

The essential characteristic of the heating element is a large positive temperature coefficient of resistance This property dramatically increases the element resistance as the temperature rises, resulting in effectively limiting the temperature.

Another essential characteristic is that the surface temperature of the heating pad assembly held against the skin is approximately 101 F. degrees. Temperatures over 101 degrees can induce undesirable sweating under the pad. A heated seat pad designed for outdoor use operates through heavy clothing, thus may be operated at temperatures as high as 110 F. degrees. Higher heating element temperatures may be used when adequate isolation between the heater and skin is used to prevent scalding.

In contrast to the common 110 volt heating pads, which wastefully dissipate heat from both surfaces, the present invention is heavily insulated on the distal side, thus substantially all the thermal energy generated is transferred to the wearer. The low temperature and efficiency of directing the heat to the user, results in a substantial reduction of power required to operate usefully. Outdoor seat cushion applications operate successfully on as little as 4 watts. 4 watts is approximately 3.4 food calories/hour.

Panels or sheets of conductive polymer have other properties useful within the scope of the present invention. One such property is that the material is tolerant to perforation. Thus the sheets may be perforated with holes or slots to permit the passage of air and moisture. Perforate-ability also permits assembly by sewing, riveting, attachment of snaps, or other penetrating fastening as an alternative to adhesives. Another characteristic of heating elements constructed of conductive polymer material is that the heat is produced over the entire current carrying surface between the contact electrodes. This is in contrast to serpentine wire elements, which produce heat only along the thin line of the wire. A single break in the wire will disable the heating element. Not so, in the case of a polymer sheet heating element; small breaks affect only small areas of such an element.

### BRIEF DESCRIPTION OF THE FIGURES

FIG. 1 is a belt and heating system adapted for use on the lumbar region of the back.

FIG. 2 is a cutaway view of the heating pad portion of the heated lumbar belt.

FIG. 3 is a cutaway view of the heated seat cushion.

### TABLE OF IDENTIFIED DETAILS

1. Lumbar warming system
2. Back segment
3a & 3b Pockets for heating pad and battery pack
4. Heating pad
5. Battery pack
6. Elastic portion of belt
7a & 7b Closing system, buckle or other means.
8. Battery charger
9. Connector, charger to battery
10. Connector, battery to heating pad
11. Connector, heating pad to battery
12. Not assigned
13. Overall cover.
14. Heating element.
15. Distal side insulation.
16a & 16b Electrodes to connect power to the heating resistor.
100. Heated seat cushion
102. Cushion padding
104. Insulation layer or panel
106. Heating element
108. Cushion sub-cover
110. Outer cover, cushion case
112. Wiring to heating element.
114. Wiring to battery pack.
116. Electrical connector to heating element.
118. Electrical connector to battery pack.
120. Battery pack
122. Carrying strap
126 Cover flap
128 & 129. Velcro flap closing latch
130. Battery charger.
132. Electrical connection and wiring to connect charger to battery pack.

### DETAILED DESCRIPTION OF THE INVENTION

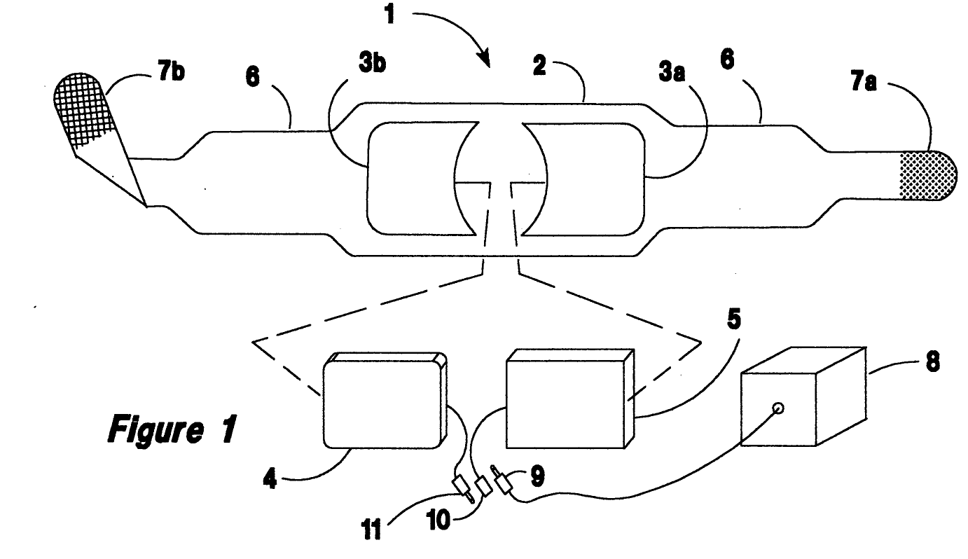

FIG. 1 illustrates an embodiment of the invention 1 particularly adapted for use as a heated belt for the lumbar region of the spine. The belt is comprised of a back portion 2 fitted with pockets 3a and 3b for receiving the heating module 4 and battery pack 5. The belt further comprises elastic segments 6, and a buckle system 7. The preferred buckle system is hook and loop fasteners (Velcro). However, since the buckle system is merely for closing the belt into a loop, other methods of connection such as hole-and-tang buckles, snaps, buttons, ties, etc are equivalent and will work just as well. Obviously, the pocket for the heated pad may span the entire back or one or more hot pads may be inserted in the pocket or pockets. The battery pack may then carried elsewhere on or off the support belt.

The heated pad and battery are interconnected by wiring through connectors 10 and 11. The connectors also function as an on-off switch. Making the connection requires specific actions and is visible, thus the system cannot accidentally be switched on. Alternatively, a conventional switch may be installed to turn the electric power off and on.

The preferred heating element from Electro-Plastics has at least two metallic contacting conductors (electrodes) 16a & 16b disposed along or within opposite edges of the conductive polymer sheet. The current flows from edge to edge. More electrodes may be used in alternating polarities, thereby dividing the heater into zones and providing other electrical advantages.

The battery is maintained by a battery charger 8 with connector 9 A Y adapter (not shown) capable of connecting 9, 10 and 11 together in any combination permits operating the heater directly off the charger or battery, or to operate the heater while charging the battery.

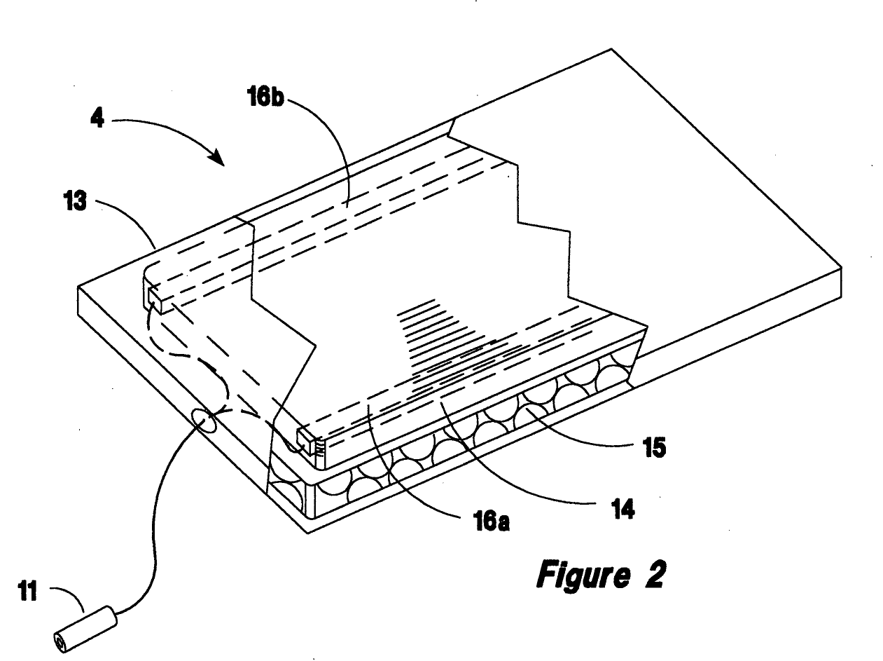

FIG. 2 shows details of the spot heating pad 4 as used in the lumbar warming belt and other heating applications.

Conductive sheeting heating element 14 is backed by a layer of insulation material 15 to prevent heat loss in the direction away from the user. The preferred insulation material is two sheets of bubble wrap film laid with the bubbles together. Bubble wrap film has good insulation properties, is flexible, tough, and inexpensive. Other insulating materials may be used with equal success for insulating against heat passage and consequence loss. The heating pad 14 and insulation 15 is encased in a conforming cloth or plastic case 13. Other layers of material may be added to provide stiffness or conformance to a specific shape when such characteristics may be needed. Similarly, the heater 14, insulation 15, and cover 13 may be attached to each other by any reasonable fastening means such as glueing, sewing, etc. An optional thermally reflective layer may be placed between the heating element and the insulation layer to augment the insulation provided by the insulation layer.

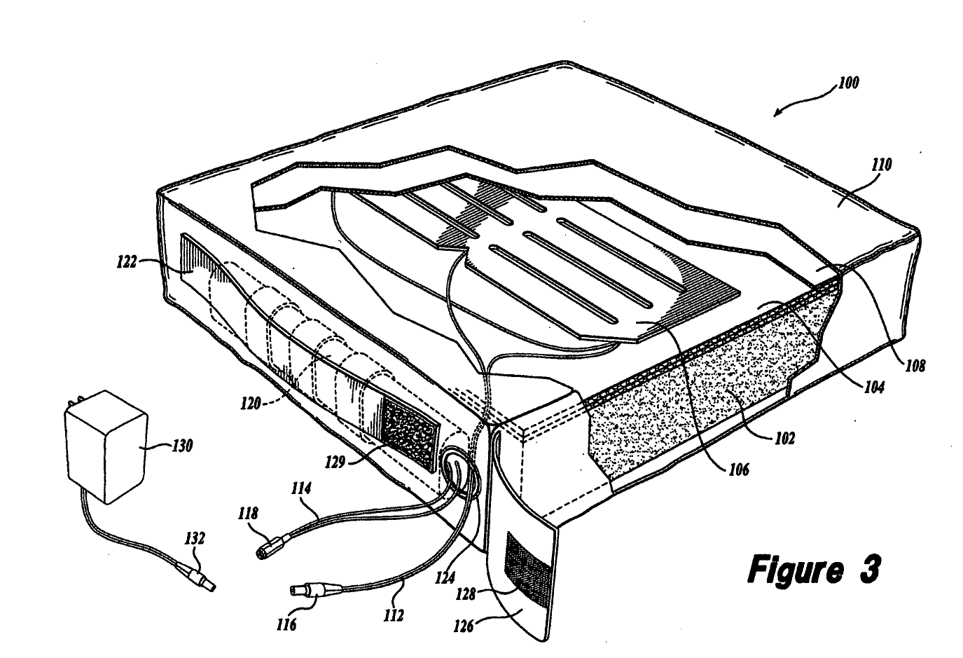

FIG. 3 illustrates a seat cushion designed for outdoor use in stadiums, small boats, and other seated activities.

In FIG. 3, the heated cushion 100 comprising a resilient foam core 102 that is generally in the form of a rectangular, circular, oval or other shape. A rectangular pad is preferred to be approximately 14 inches square by 1.5 inches thick. The foam pad provides softness for comfort and may also be designed as a floatation device for boating. The insulating layer may further comprise an optional thermally reflective panel or layer 104 of metal foil, metalized Mylar, or the like is disposed above and substantially covers a top surface of the foam core 102. A heating element 106, preferably made from a conductive polymer blend, is generally centrally disposed on top of the thermally insulating layer 104. The heating element 106 is a thin and flat flexible panel having a significant positive thermal coefficient (PTC) of resistance which has an advantage of being self-regulating, i.e., it will not exceed a particular design temperature. Therefore, no separate temperature modulation unit is required.

It will be appreciated that the bubble wrap material of the thermally insulating panel 104 provides a degree of thermal insulation between the foam core 102 and the heating element 106 and, therefore, cooperatively with an optional reflective foil, metalized Mylar sheet, or similar preferentially directs the thermal energy from the heating element 106 upwardly, toward the user. It is contemplated that additional insulating layers between the foam core 102 and the heating element 106 may also be provided, and it is recognized that the foam core 102 itself has significant insulating properties. It is obvious that other materials may be substituted for bubble wrap to provide adequate insulation to prevent heat from escaping in the direction away from the user.

A heating element cover 108 is disposed over the heating element 106. In the disclosed embodiment, the heating element cover 108 is an elastic sheet, such as neoprene, larger than the heating element 108, having an adhesive lower face. The heating element cover 108 therefore secures the heating element 106 in its generally central position to the thermally insulating panel 104. The heating element 106 is therefore protected and secured between the panel 104 and the heating element cover 108. An outer cover 110, which may be made from a plastic or natural fabric material, wraps about and provides an outer holder for the foam core 102, insulating panel 104, heating element 106, and heating element cover 108. In the disclosed embodiment, the cover 110 is of sewed construction and snugly accommodates the other components discussed above. The outer cover 110 preferably includes a convenient carry strap 122 along at least one edge. It is preferred that the outer cover be provided with a texture or other attribute to increase the friction force between the user and the cover 110 to reduce slipping and, therefore, increase the user's comfort. One such friction increasing means is found as an intrinsic property of vinyl “ink” used to print logos and images on fabric. Other friction increasing means are known in the art.

A battery module 120 is disposed along one side of the foam core 102 and within the outer cover 110. The battery module 120 of the currently preferred embodiment of the present invention is a rechargeable 14.4 V, 2Ah Nickel-Cadmium or Nickel-Hydride battery pack, as is known in the art. It will be appreciated, however, that other battery modules or other portable power sources may alternatively be used, including, for example, high capacity rechargeable and non-rechargeable batteries, power supplies, fuel cell modules and the like.

For outdoor use, a cushion should provide some tactile warmth to augment and help support overall body temperature regulation. Therefore, the heater should operate at above 99 degrees, preferably about 110 F. degrees. Temperature set point is most easily regulated by varying the voltage impressed across the conductive portions of the heater element.

Although the cushion of FIG. 3 is fashioned particularly for sitting upon, it does make a very good back, leg, and abdomen warmer, and a quite satisfactory warmed lap desk for writing or operating a lap-top computer.

### Alternative Embodiments and Variations of the Invention

A layer of porous, wicking type fabric well known in the outdoor wear industry may be placed between the heating pad and the wearer's skin to permit the skin to “breathe”. The reduced thermal conductivity of such a layer will require, and will automatically induce, the heater to be operated at a slightly higher temperature if the same tactile warmth is to be maintained.

The self-regulating characteristic of the preferred heater material will automatically provide a modest increase in temperature.

While the description of the invention presented here has been centered on two embodiments, a battery operated seat cushion and a heated pad with a pad holder (belt) for snuggling a heater pad against a body in the lumbar region, the disclosed and claimed invention is intended to include similar structures of heater and holder for warming other parts of the body, furniture, and larger structures such as benches.

Clothing and specialized apparel such as jackets, vests, cummerbunds, boot inserts, scuba suits, survival suits, shawls, scarves, muffs, gloves, hats, blankets, etc are anticipated to be fitted with the described body warming invention without departing from the spirit of this disclosure.

Of particular note is the use of the present invention with suitable modifications of the fastening means and size to warm the legs of athletes waiting their turn on the sideline benches. The heating pads are light enough to be worn on the field, or may be quickly removed when the player is called to the field. He should be more ready to perform strenuously as required by his sport.

The protocol for treatment of hypothermia is to not apply heat to the skin as that causes blood to flow and carry chilled blood from the cold outer tissues into the center of the body. The present invention should be highly desirable in the field treatment of hypothermia. A blanket or heating pad operating at the temperature of the skin is equivalent to a blanket with an infinite “R” value. No heat is lost from the body and no heat is passed to the body. This permits heating of the outer body layers from within. Also the effect of no heat loss is a sensation of warmth caused by body warmth coming to the skin from within.

The spot heaters described can be placed on locations where the veins and arteries are near the surface such as the backs of the knees, elbows, wrists, groin, and neck. The gentle heat thus applied will be carried almost directly to the interior of the body which is the preferred zone to be warmed first.

For more precise temperature control, the resistance of the heating element can be measured using any of several well known means. Since the resistance of the element and the element's temperature are closely related and repeatable, the energizing power may be modulated with the result of temperature control with more precision than available from simply using the change of resistance to directly limit the current through the heating element.

This disclosure has centered on describing the power source as portable battery packs. It is obvious that larger batteries, and other power packs and power supplies that operate off the commercial mains, automotive, marine, and aircraft system power are suitable when true mobility is not an issue of use. Because the heating element is entirely ohmic, that is, passive resistance, the heating element will operate on DC, AC, pulsed, rectified AC, etc electrical power.

### How to Use the Invention

The invention as described in detail and as extrapolated to other similar uses is placed as close to the skin as possible with the insulating side outward from the skin, and energized from the power source.

The heating element quickly warms and blocks heat flow from the wearer's skin. If it the heating element warms to over 99 degrees F. Heat will pass from the element to the wearer's skin. The temperature will continue to rise, which increases the element's resistance, which reduces the current flowing and thus reducing the thermal energy produced. Eventually, the temperature reaches a degree where the heat energy flow to the skin (and minimal flow to the environment) balances the electrical energy supplied by the power source, and the heater temperature stops rising.

While the preferred embodiment of the invention has been illustrated and described, it will be appreciated that various changes can be made therein without departing from the spirit and scope of the invention.

I claim:

1. An electrically heated pad for applying heat to the skin of a living animal comprising:
a. a flexible heating element comprising a sheet of conductive polymer material having a proximal side and a distal side, a significant positive thermal coefficient of resistance, and at least one pair of cooperating electrodes for applying electrical energy to the sheet, and
b. a thermally insulating layer disposed on the distal side of said heating element for preventing loss of heat energy from the distal side, and
c. a flexible cover enclosing the heating element and the insulating layer; and
d. a power supply electrically connected to the said electrodes of the flexible heating element to provide electrical energy to said heating element.

2. The heated pad of claim 1, wherein the animal is a human person, a domestic livestock, a zoological specimen.

3. The heating pad of claim 1 where the said thermal insulating layer further comprises a reflective layer.

4. The heating pad of claim 1 further comprising a flexible cushion disposed between said insulating layer and the distal side of said enclosing cover.

6. The heating pad of claim 1 wherein insulating layer comprises at least one layer of bubble wrap material.

7. The heating pad of claim 1, wherein the heating element is positioned to preferentially direct heat toward the said animal, and there is no insulation disposed between the said heating element and said element.

8. The heating pad of claim 1 wherein the operating temperature of the heating element is between 99 and 110 degrees Fahrenheit.

9. The heating pad of claim 1, where the positive thermal coefficient of resistance of said heating element is sufficient to establish temperature regulation when a constant voltage is applied to the heating element.

10. The heating pad of claim 1, wherein the portable power supply is a rechargeable battery pack.

11. The heating pad of claim 10, further comprising a disconnectable power supply for recharging the rechargeable battery pack.

12. A heated cushion comprising: a foam core having an upper surface; an insulating panel disposed on the upper surface of the foam core; a flexible heating element having PTC properties disposed on the insulating layer; a cover panel disposed over the flexible heating element; a portable power supply electrically connected to the flexible heating element; and a flexible cover enclosing the foam core, the thermally insulating panel, the flexible heating element, the cover panel, and the portable power supply.

13. The heated cushion of claim 12, wherein the cover panel comprises a sheet of neoprene.

14. The heated cushion of claim 12, wherein the thermally insulating panel includes an upwardly reflective layer.

15. The heated cushion of claim 12, wherein the thermally insulating layer comprises a bubble wrap material.

16. The heated cushion of claim 12, wherein the portable power supply is a rechargeable battery pack.

17. The heated cushion of claim 16, further comprising a removable power supply that recharges the rechargeable battery pack.

18. The heated cushion of claim 12, wherein the flexible cover comprises a fabric material sized to snuggly accommodate the foam core, the thermally insulating panel, the flexible heating element, the cover panel, and the portable power supply.

19. The heated cushion of claim 18, wherein at least a portion of an outer surface of the flexible cover is textured to inhibit slipping.
